(19) United States
(12) Patent Application Publication
(10) Pub. No.: US 2017/0334820 A1
(43) Pub. Date: Nov. 23, 2017

(54) USE OF 3,3'-DIMETHOXY-4,4'-DIHYDROXYSTILBENES AS A FLAVORING SUBSTANCE

(71) Applicant: BASF SE, Ludwigshafen (DE)

(72) Inventors: Ralf PELZER, Fürstenberg (DE); Carolin REGENBRECHT, Stuttgart (DE)

(21) Appl. No.: 15/522,583

(22) PCT Filed: Nov. 6, 2015
(86) PCT No.: PCT/EP2015/075868

§ 371 Date: Apr. 27, 2017

(30) Foreign Application Priority Data
Nov. 7, 2014 (EP) 14192281.5

(51) Int. Cl. C07C 41/36; C07C 43/23; C11B 9/00; C11B 9/02

(52) U.S. Cl. CPC C07C 41/36; C11B 9/025; C07C 43/23; C11B 9/0061

(57) ABSTRACT

The present invention relates to the use of 3,3′-dimethoxy-4,4′-dihydroxystilbene as an odorous substance, in particular for developing a vanilla odor under the action of light. Furthermore, the invention relates to a composition which contain 3,3′-dimethoxy-4,4′-dihydroxystilbene and additionally a carrier, fragrance compositions and/or an odorant material which contain said compound and a process for imparting or modifying an odor of compositions by adding said compound to these compositions. The invention additionally relates to a process for obtaining 3,3′-dimethoxy-4,4′-dihydroxystilbene from lignin-containing compositions.

The present invention relates to the use of 3,3′-dimethoxy-4,4′-dihydroxystilbene as an odorous substance, in particular for developing a vanilla odor under the action of light. Furthermore, the Invention relates to a composition which contain 3,3′-dimethoxy-4,4′-dihydroxystilbene and additionally a carrier, fragrance compositions and/or an odorant material which contain said compound and a process for imparting or modifying an odor of compositions by adding said compound to these compositions. The invention additionally relates to a process for obtaining 3,3′-dimethoxy-4,4′-dihydroxystilbene from lignin-containing compositions.

### BACKGROUND OF THE INVENTION

Odorous substances are of great commercial interest, particularly in the field of cosmetics and as an additive in washing powders and cleansing agents. However, the obtention of fragrances from natural sources is mostly costly and the quantities thus obtainable are limited. In addition, the purity or production quantity of these fragrances often varies because of variable environmental conditions in the production of the raw materials from which these are isolated. There is therefore great interest in finding new odorous substances which can be obtained from readily accessible natural sources which are available in large quantities.

Often in the search for odorous substances chemical compounds are of interest which on the basis of their characteristic odor can be used as a substitute for natural substance, with substitute and natural substance not necessarily having to display a chemical structural similarity.

Since small changes in the chemical structure already cause massive changes in the sensory properties such as odor and also taste, the targeted search for substances with defined sensory properties, such as a defined odor, becomes extremely difficult. The search for novel odorous substances is therefore mostly tedious without knowing whether a substance with the desired odor is even actually found.

Hydroxystlibene derivatives such as (E)-3,3′-dimethoxy-4,4′-dihydroxystilbene are in principle known. For example, Dhyani et al., Applied Radiation and Isotopes, 2011, Vol. 69, pp 996-1001, describe the stereoselective synthesis of (E)-3,3′-dimethoxy-4,4′-dihydroxystilbene by a McMurry cross-coupling reaction starting from vanillin.

Apart from the synthetic process, it is also known that (E)-3,3′-dimethoxy-4,4′-dihydroxystilbene can be obtained from natural sources, since this occurs naturally in plants.

Hajdú et al., J. Nat. Prod., 1998, Vol. 61, pp. 1298-1299, describe the extraction, isolation and characterization of (E)-3,3′-dimethoxy-4,4′-dihydroxystilbene from Leuzea carthamoides (Asteraceae).

Furthermore, (E)-3,3′-dimethoxy-4,4′-dihydroxystilbene is for example contained in extracts and the syrup from maple trees, in particular the sugar maple—see WO 2012/021981, WO 2012/021983 A1 and WO 2012/055010 A1. These documents inter alia describe the potential use of these extracts and syrups, which as well as (E)-3,3′-dimethoxy-4,4′-dihydroxystilbene also contain many other secondary plant substances, in the cosmetic and pharmaceutical field and in foods.

### SUMMARY OF THE INVENTION

The present invention is based on the objective of providing novel odorous substances which can be obtained from readily available natural sources or can be synthesized on a large industrial scale from readily obtainable educts. Compounds are preferably sought which possess advantageous sensory properties, i.e. an intense, pleasant odor, in particular a vanilla-like odor. The novel odorous substances should also be toxicologically harmless.

It was surprisingly found that the compound 3,3′-dimethoxy-4,4′-dihydroxystilbene (I)

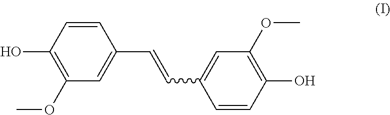 [SMILES: COc1cc(C=Cc2ccc(O)c(OC)c2)ccc1O]

possesses a vanilla-like odor and has the desired advantageous sensory properties. Furthermore, it was surprisingly found that the typically vanilla-like odor of 3,3′-dimethoxy-4,4′-dihydroxystilbene (I) intensifies under the action of sunlight. Moreover, 3,3′-dimethoxy-4,4′-dihydroxystilbene (I) can be obtained in large quantities from lignin-containing compositions.

The present invention thus relates to the use of 3,3′-dimethoxy-4,4′-dihydroxystilbene as an odorous substance.

Furthermore the present invention relates to the use of 3,3′-dimethoxy-4,4′-dihydroxy-stilbene for developing a vanillin odor under the action of light.

Furthermore the present invention relates to the use of 3,3′-dimethoxy-4,4′-dihydroxy-stilbene as a component of a composition which additionally contains a carrier, wherein the composition is selected from washing powders, laundry conditioners, cleansing agents, fragrance-containing hygiene products, fragrance dispensers and perfumes.

Furthermore the present invention relates to a fragrance composition and/or an odorant material containing 3,3′-dimethoxy-4,4′-dihydroxystilbene and a carrier, wherein the fragrance composition and/or the odorant material contains the relevant 3,3′-dimethoxy-4,4′-dihydroxystilbene in a quantity which imparts an odor to the fragrance composition and/or the odorant material or modifies the odor of the fragrance composition and/or the odorant material.

Furthermore the present invention relates to a process for imparting or modifying an odor of a composition, in which 3,3′-dimethoxy-4,4′-dihydroxystilbene is added to the composition in a quantity which imparts an odor to the composition or modifies the odor of the composition.

Furthermore the present invention relates to a process for obtaining 3,3′-dimethoxy-4,4′-dihydroxystilbene from aqueous, alkaline lignin-containing compositions, in which the aqueous, alkaline lignin-containing composition, which has optionally been treated with alkalis or oxidatively, is treated with a solid adsorbent, the adsorbent is separated from the aqueous, alkaline lignin-containing composition and then to obtain the 3,3′-dimethoxy-4,4′-dihydroxystilbenes (I) the adsorbent is treated with an organic solvent, whereby a 3,3′-dimethoxy-4,4′-dihydroxystilbene-containing eluate is obtained.

3,3′-dimethoxy-4,4′-dihydroxystilbene is characterized by its advantageous organoleptic properties, in particular by a pleasant vanilla-like odor. 3,3′-dimethoxy-4,4′-dihydroxy-stilbene can therefore advantageously be used as an odorous substance or as a component of a fragrance composition and/or an odorant material.

3,3′-dimethoxy-4,4′-dihydroxystilbene is in particular characterized in that the typical vanilla-like odor intensifies under the action of sunlight. In addition, 3,3′-dimethoxy-4,4′-dihydroxystilbene can be applied very well onto textiles or textile fibers and adheres to these for a long time.

Because of its physical properties 3,3′-dimethoxy-4,4′-dihydroxystilbene possesses very good, practically universal solvent properties for other odorous substances or other commercially obtainable ingredients such as are used in fragrance compositions, in particular in perfumes.

3,3′-dimethoxy-4,4′-dihydroxystilbene can both be obtained from readily accessible natural sources and also be synthesized from cheap and readily accessible starting materials.

3,3′-dimethoxy-4,4′-dihydroxystilbene is probably of very low toxicity, since this compound belongs to a group of secondary plant substances which display no significant toxicity. For example, the structurally very similar compound resveratrol, which has been extensively studied pharmacologically and is classified as toxicologically harmless, belongs to this group (Cottart et al., Mol. Nutr. Food Res., 2010, Vol. 54(1), pp. 7-16).

### DETAILED DESCRIPTION OF THE INVENTION

Because of the double bond, the compound 3,3-dimethoxy-4,4′-dihydroxystilbene (I)

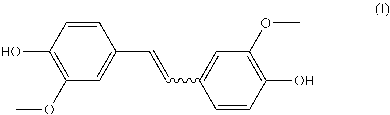 [SMILES: COc1cc(C=Cc2ccc(O)c(OC)c2)ccc1O]

can exist as the E isomer (E)-3,3′-dimethoxy-4,4′-dihydroxystilbene (I-E) or as the Z isomer (Z)-3,3′-dimethoxy-4,4′-dihydroxystilbene (I-Z)

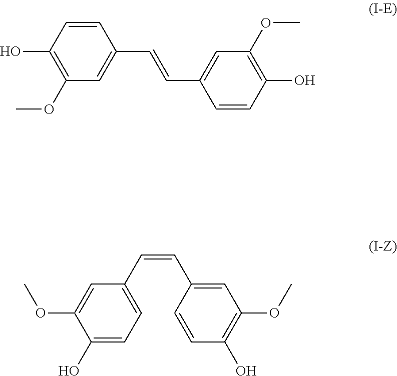

or as an E/Z isomer mixture.

The present invention thus relates to both the use of the E isomer and also the use of the Z isomer and the use of mixtures thereof. The expression “3,3′-dimethoxy-4,4′-dihydroxystilbene” comprises both the pure E and the pure Z isomer and also mixtures in which the isomers are present in equal quantities or contains one of the Isomers in excess.

In a preferred embodiment of the invention, the compound (I) is present as pure E isomer or as an E/Z isomer mixture which predominantly contains the E isomer (I-E). To be more precise, the compound (I) is present as pure E isomer or as an E/Z isomer mixture which contains at least 60 wt. %, in particular at least 80 wt. % and especially at least 90 wt. % of the E isomer (I-E), based on the total quantity of the isomers I-E and I-Z.

The compound (I) in a great variety of states of purity is suitable for use as an odorous substance. For use according to the invention as an odorous substance, the purity of the compound (1) is therefore not specifically limited. Preferably, the compound (I) has a purity of at least 50%, in particular at least 80% and especially at least 90%.

The aforesaid preferred embodiments can be combined with one another as desired.

In a preferred embodiment of the invention, the compound (I) accordingly has a purity of at least 90%, where this is present as pure E isomer or as an E/Z isomer mixture which contains at least 90 wt. % of the E isomer (I-E).

As already mentioned, 3,3′-dimethoxy-4,4′-dihydroxystilbene has advantageous sensory properties, in particular a pleasant odor. To be specific, 3,3′-dimethoxy-4,4′-dihydroxystilbene possesses an evocative vanilla-like odor, which is comparable with that of vanillin or ethylvanillin.

It was suprisingly found that the evocative vanilla-like odor of 3,3′-dimethoxy-4,4′-dihydroxystilbene intensifies through the action of light. For this reason, the invention also relates to the use of the compound (I), as defined above, for developing a vanilla-like odor under the action of light.

In the context of the present invention the term “action of light” is understood to mean a procedure in which the compound (I) is exposed to the light from an artificial light source or sunlight. As a rule, intensification of the vanilla-like odor already occurs after brief exposure to light, wherein an exposure duration in the range from 1 to 60 minutes or 1 to 30 minutes suffices to achieve an appreciable intensification of the vanilla-like odor. Of course, longer exposure durations can also lead to significant intensification of the vanilla-like odor.

Suitable artificial light sources can be light sources the emitted light wherefrom includes the spectral region of visible light and also the spectral regions adjacent thereto, or also light sources which emit light of a specific region of visible light and the spectral regions adjacent thereto.

Suitable artificial light sources are for example heat radiators such as incandescent lamps, gas discharge lamps such as mercury vapor high pressure lamps, sodium vapor high pressure lamps, xenon gas discharge lamps or halogen-metal vapor lamps and light-emitting diodes.

In a preferred embodiment of the invention, for the development of a vanilla-like odor the compound (I) is exposed to sunlight for a defined time period, for example 1 to 60 minutes, in particular 1 to 30 minutes.

Intensive odor impressions should be understood to mean those properties of aroma chemicals which make evocative perception already possible at very low head space concentrations. The intensity can be determined via a threshold value determination. A threshold value is that concentration of a substance in the relevant head space at which an odor impression is still just perceived by a representative test panel, where this does not however have to be further defined. One substance class which is probably is among the most intensely odorous substance classes, i.e. has very low odor threshold values, are thiols, whose threshold value often lies in the ppb/m³ range. The purpose of the search for novel aroma chemicals is to find substances with as low as possible an odor threshold, in order to enable as low as possible a use concentration. The more closely this target is approached, the more they are described as “intense” odor substances or aroma chemicals.

“Advantageous sensory properties” or “pleasant odor” are hedonistic expressions which describe the beauty and evocation of an odor impression conveyed by an aroma chemical.

“Beauty” and “evocation” are terms which are familiar to those skilled in the art, to a perfumer. Beauty as a rule refers to a spontaneously evoked, positively experienced, present sensory impression. However, “beautiful” does not have to be synonymous with “sweet”. The odor of musk or sandalwood can also be “beautiful”. “Evocation” as a rule refers to a spontaneously evoked sensory impression which—in the same test panel—evokes a reproducibly similar memory of something specific.

For example, a substance can have an odor which is spontaneously reminiscent of that of an “apple”: the odor would then be evocative of “apple”. If this odor of apple were very pleasant, because the odor for example is reminiscent of a sweet, fully ripe apple, the odor would have to be called “beautiful”. However, the odor of a typically sour apple can also be evocative. If both reactions occur on smelling the substance, i.e. for example a beautiful and evocative odor of apple, then this substance displays especially advantageous sensory properties.

Furthermore the present invention relates to the use of 3,3′-dimethoxy-4,4′-dihydroxy-stilbene, as defined above, as a component of a composition which typically contains at least one aroma substance, i.e. odorous substance and additionally a carrier. Such compositions are for example selected from washing powders, laundry conditioners, cleansing agents, fragrance-containing hygiene products, such as diapers, sanitary napkins, armpit pads, paper tissues, wet wipes, toilet paper, handkerchiefs and the like, and fragrance dispensers such as air fresheners and perfumes.

For the formulation of these compositions, 3,3′-dimethoxy-4,4′-dihydroxystilbene, as defined above, optionally together with one or more other aroma substances, is usually added to an existing preparation which previously contains no aroma substances or one or more aroma substances different from the compounds (I). Usually these compositions additionally contain a carrier, which can consist of one compound, a mixture of compounds or other additives, which have no or no appreciable sensory properties. However, the carrier can also be a compound or an additive which has appreciable sensory properties, or be a mixture of compounds which contains at least one aroma substance different from the compounds (I) and optionally at least one further compound which possesses no appreciable sensory properties.

The carrier can be a compound, a mixture of compounds or other additives which have the aforesaid properties. Suitable carriers include liquid or oily carriers and waxy or solid carriers.

Suitable liquid or oily carriers are for example selected from alcohols such as ethanol, water, aliphatic diols and polyols with a melting temperature below 20° C., such as ethylene glycol, glycerol, diglycerol, propylene glycol or dipropylene glycol, cyclic siloxanes such as hexamethylcyclotrisiloxane or decamethylcyclopentasiloxane, plant oils such as fractionated coconut oil or esters of fatty alcohols with melting temperatures below 20° C., such as tetradecyl acetate or tetradecyl lactate, and alkyl esters of fatty acids with melting temperatures below 20° C., such as isopropyl myristate.

Suitable waxy or solid carriers are for example selected from fatty alcohols with melting temperatures above 20° C., such as myristyl alcohol, stearyl alcohol or cetyl alcohol, polyols with melting temperatures above 20° C., fatty acid esters with fatty alcohols which have a melting temperature above 20° C., such as lanolin, beeswax, carnauba wax, candelilla wax or Japan wax, waxes produced from petroleum, such as solid paraffin, water-insoluble porous minerals such as silica gel, silicates, for example talc, microporous crystalline alumosilicates (zeolites), clay minerals, for example bentonite, or phosphates, for example sodium tripolyphosphate, paper, cardboard, wood, and textile composite or nonwoven materials from natural or synthetic fibers.

Suitable carriers are for example also selected from water-soluble polymers, such as polyacrylate esters or quaternized polyvinylpyrrolidones, or water-alcohol-soluble polymers such as specific thermoplastic polyesters and polyamides. The polymeric carrier can be in various forms, e.g. in the form of a gel, a paste, as solid particles, such as microcapsules, or brittle coatings.

As a rule, the quantities of 3,3′-dimethoxy-4,4′-dihydroxystilbene, as defined above, used in these compositions correspond to the common, normal commercial use quantities for fragrances in formulations. To be more precise, the quantity of 3,3′-dimethoxy-4,4′-dihydroxystilbene, as defined above, used lies in the range from 0.001 to 50 wt. %, in particular in the range from 0.01 to 20 wt. % and especially in the range from 0.1 to 10 wt. %, based on the total weight of the composition.

In a particularly preferred embodiment of the present invention, 3,3′-dimethoxy-4,4′-dihydroxystilbene, as defined above, is used as a component in compositions, which on the basis of their use as intended are at least to some extent exposed to sunlight, such as for example in washing powders, laundry conditioners, cleansing agents or fragrance-containing hygiene products.

In a particularly preferred embodiment of the present invention, 3,3′-dimethoxy-4,4′-dihydroxystilbene, as defined above, is used as a component in washing powders and laundry conditioners. A particularly advantageous effect here results from the fact that the compound (I) continues to adhere very well to natural, synthetic or semisynthetic fiber materials which are usually used for the production of textiles. These fiber materials can in principle contain all natural fibers and/or chemical fibers which can be processed in textile manufacturing processes, or consist of these.

The natural fibers suitable for textile production are for example plant fibers such as cotton fibers, flax fibers or hemp fibers, or animal fibers such as wool, cashmere or silk.

The chemical fibers suitable for textile production are for example fibers of natural polymers such as viscose, lyocell or rubber, or fibers of synthetic polymers such as polyester, polyacrylonitrile, polypropylene or polyamide.

In the context of the present invention, washing powders and laundry conditioners are understood to mean agents which are used for cleaning flexible materials with high absorbency, e.g. of materials of a textile nature.

Examples of flexible materials with high absorbency are those which contain natural, synthetic or semisynthetic fiber materials, as previously defined, or consist thereof and which accordingly as a rule at least to some extent have a textile nature. The materials containing fibers or consisting of fibers can in principle be in any form occurring in use or production and processing. For example fibers can be present disordered in the form of flock or pile, ordered in the form of threads, yarns, twisted yarn or in the form of flat sheets such as fleeces, Loden materials or felt, fabrics, and knitted fabrics in all possible weave types. The fibers can be raw fibers or fibers at any processing stages.

Owing to the fact that the compound (I) continues to adhere very well to natural, synthetic or semisynthetic fiber materials, the quantity of 3,3′-dimethoxy-4,4′-dihydroxystilbene used as an odor-imparting component in washing powders and laundry conditioners which is necessary in order to achieve the desired odor effect is as a rule relatively low. To be more precise, the quantity of 3,3′-dimethoxy-4,4′-dihydroxystilbene, as defined above, used in washing powders and laundry conditioners usually lies in the range from 0.0001 to 20 wt. %, in particular in the range from 0.001 to 10 wt % and especially in the range from 0.01 to 5 wt %, based on the total weight of the composition.

Depending on their use purpose, the compositions in which 3,3′-dimethoxy-4,4′-dihydroxystilbene, as defined above, is used as an odor-imparting component can contain other auxiliary substances and/or additives, such as for example detergents or mixtures of detergents, thickeners such as polyethylene glycols with a number average molecular weight from 400 to 20,000 Da, lubricants, binders or agglomerants such as sodium siliciate, dispersants, builder salts, water softeners, filler salts, pigments, colorants, optical brighteners, soil dispersants and the like.

In particular washing powders and laundry conditioners as a rule contain several specific additives such as for example surfactants, water softeners, builders, wash alkalis, enzymes, soil dispersants, defoamants, suspending agents, bleaching agents or optical brighteners.

Furthermore the present invention relates to a fragrance composition and/or an odorant material containing 3,3′-dimethoxy-4,4′-dihydroxystilbene or an E/Z isomer mixture thereof, as defined above, and a carrier, wherein the fragrance composition and/or the odorant material contains the particular 3,3′-dimethoxy-4,4′-dihydroxy-stilbene, as defined above, in a quantity which imparts an odor to the fragrance composition and/or the odorant material or modifies the odor of the fragrance composition and/or the odorant material.

The quantities of 3,3′-dimethoxy-4,4′-dihydroxystilbene required for this depend on the nature and the use purpose of the fragrance composition and/or odorant material and can therefore vary greatly.

Apart from this, the total concentration of 3,3′-dimethoxy-4,4′-dihydroxystilbene in the fragrance composition according to the invention and/or the odorant material according to the invention is not specifically limited and can be adapted over a wide range to the particular use purpose. As a rule, the common normal commercial use quantities for fragrances can be used. Usually, the total quantity of 3,3′-dimethoxy-4,4′-dihydroxy-stilbene in the fragrance composition according to the invention and/or the odorant material according to the Invention lies in the range from 0.0001 to 20 wt. % and in particular in the range from 0.001 to 10 wt. %.

Typical use fields for the fragrance compositions and/or odorant materials according to the invention are washing powders, textile conditioners, cleansing agents, preparations of fragrances for the human or animal body, for rooms such as kitchen, wet areas, cars or trucks, for natural or artificial plants, for clothing, for shoes and shoe insoles, for items of furniture, for carpets, for air humidifiers, for air perfumers or for perfumes.

In particular, the fragrance compositions and/or odorant materials according to the invention are used in washing powders, textile conditioners and in fragrance preparations for clothes, shoes, furniture or carpets.

The invention also includes odorant combinations which contain 3,3′-dimethoxy-4,4′-dihydroxystilbene, as defined above, as component A and at least one further compound known as an odorous or aroma substance as component B, such as for example one or more of the following compounds B1 to B11:

B1: Methyl dihydrojasmonate (e.g. Hedione),
B2: 4,6,6,7,8,8-hexamethyl-1,3,4,6,7,8-hexahydrocyclopenta[g]benzopyran (e.g. Galaxolide™),
B3: 2-methyl-3-(4-tert-butylphenyl)propanal (Lysmeral™),
B4: 2-methyl-3-(4-iso-propylphenyl)propanal (cyclamen aldehyde),
B5: 2,6-dimethyl-7-octen-2-ol (dihydromyrcenol),
B6: 3,7-dimethyl-1,6-octadien-3-ol (linalool),
B7: 3,7-dimethyl-trans-2,6-octadien-1-ol (geraniol),
B8: 2,3,8,8-tetramethyl-1,2,3,4,5,6,7,8-octahydro-2-naphthalenyl methyl ketone (Iso E Super™),
B9: alpha-hexylcinnamaldehyde,
B10: 3,7-dimethyl-6-octen-1-ol (citronellol),
B11: alpha, or beta-, or delta-damascone.

As formulations of odorous substances, for example the formulations disclosed in JP 11-071312 A, paragraphs [0090] to [0092] are suitable. Also suitable are the formulations from JP 11-035969 A, paragraphs [0039] to [0043].

Furthermore the present invention relates to a process for imparting or modifying an odor of a composition, in which 3,3′-dimethoxy-4,4′-dihydroxystilbene, as defined above, is added to the composition in a quantity which imparts an odor to the composition or modifies the odor of the composition. The quantities of 3,3′-dimethoxy-4,4′-dihydroxystilbene required for this depend on the state and the use purpose of the composition and can therefore vary greatly. As a rule, the quantities of 3,3′-dimethoxy-4,4′-dihydroxystilbene, as defined above, used normally lie in the range from 0.0001 to 50 wt. %, in particular in the range from 0.001 to 20 wt. %, based on the total weight of the composition.

The present invention further relates to a process for obtaining 3,3′-dimethoxy-4,4′-dihydroxystilbene (I) from aqueous, alkaline lignin-containing compositions. The process according to the Invention is characterized in that the aqueous, alkaline lignin-containing composition, which has optionally been treated with alkalis or oxidatively, is treated with a solid adsorbent, the adsorbent is separated from the aqueous, alkaline lignin-containing composition and then the adsorbent is treated with an organic solvent to obtain the 3,3′-dimethoxy-4,4′-dihydroxystilbene (I). An eluate is thereby obtained which contains the 3,3′-dimethoxy-4,4′-dihydroxystilbene (I).

For the treatment of the aqueous, alkaline lignin-containing composition which has optionally been treated with alkalis or oxidatively with a solid adsorbent, for example the solid adsorbent can be added to the aqueous, alkaline lignin-containing composition. After a certain residence time, the solid adsorbent will be separated from the aqueous, alkaline lignin-containing composition. The separation can be effected by usual solid-liquid separation processes, e.g. by filtration, sedimentation or centrifugation.

Preferably, the aqueous, alkaline lignin-containing composition is passed one or more times through a bed, or fixed bed, of the solid adsorbent, for example a column packed with the adsorbent.

Preferably, for obtaining 3,3′-dimethoxy-4,4′-dihydroxystilbene (I), an aqueous, alkaline lignin-containing composition is passed through a solid bed consisting of the solid adsorbent, in particular via a column packed with the solid adsorbent.

Following the adsorption, the release of the 3,3′-dimethoxy-4,4′-dihydroxystilbene (I) bound on the solid adsorbent is effected by treatment with an organic solvent.

Before the release of the 3,3′-dimethoxy-4,4′-dihydroxystilbene (I) bound on the solid adsorbent, the adsorbent can be treated with an aqueous solution of an acid, in particular a mineral acid or an organic sulfonic acid.

Suitable mineral acids are for example hydrochloric acid, nitric acid, perchloric acid, phosphoric acid, or sulfuric acid. Suitable organic sulfonic acids are in particular methanesulfonic acid. A particularly preferred mineral acid is sulfuric acid. Preferably the aqueous solution of the acid has an acid concentration in the range from 0.01 to 10 mol kg⁻¹, preferably in the range from 0.1 to 5 mol kg⁻¹ in particular 0.1 to 2 mol kg⁻¹.

Optionally, the solid adsorbent is washed with water before and/or after the treatment with the aqueous dilute mineral acid.

Essentially, in the process according to the invention, any aqueous lignin-containing compositions can be used which have an alkaline pH, wherein the pH is as a rule at least pH 8, in particular at least pH 9 or pH 10 and especially at least pH 11 or pH 12 and can also be pH 14.

As a rule, in the process according to the invention an aqueous, alkaline lignin-containing composition which has previously been treated with alkalis or oxidatively can also be used. In the context of the present invention, this is an aqueous, alkaline lignin-containing composition which has been obtained by dissolving a lignin or lignin derivative in aqueous alkali and/or by partial oxidation, especially by electrolysis, of an aqueous, alkaline lignin-containing composition.

The lignin or lignin derivative used for the production of the aqueous, alkaline lignin-containing composition is for example selected from lignin from black liquor, Kraft lignin, lignin sulfate, lignosulfonate, alkali lignin, soda lignin, Organosolv lignin or corresponding residues which arise in an industrial process such as pulp, cellulose pulp or cellulose production, e.g. lignin from black liquor, from the sulfite process, from the sulfate process, from the Organocell or Organosolv process, from the ASAM process, from the Kraft process or from the Natural Pulping process.

Accordingly the aqueous, alkaline lignin-containing composition used is an aqueous solution or suspension which arises as a side product in an industrial process such as pulp, cellulose pulp or cellulose production, e.g. black liquor and the lignin-containing effluent streams from the sulfite process, from the sulfate process, from the Organocell or Organosolv process, from the ASAM process, from the Kraft process or from the Natural Pulping process.

The aqueous, alkaline lignin-containing composition treated with alkalis or oxidatively as a rule has a pH of at least pH 8, often at least pH 9 or pH 10, in particular at least pH 11 or pH 12.

The aqueous, lignin-containing composition, which has optionally been treated with alkalis or oxidatively, in general contains 0.5 to 30 wt. %, preferably 1 to 15 wt. %, in particular 1 to 10 wt. % of lignin, based on the total weight of the aqueous, lignin-containing composition.

The concentration of 3,3′-dimethoxy-4,4′-dihydroxystilbenes (I) in the aqueous, lignin-containing compositions typically lies in the range from 1 to 1500 mg/kg, in particular in the range from 10 to 1000 mg/kg and especially in the range from 50 to 750 mg/kg.

In a preferred embodiment of the process according to the invention, an aqueous lignin-containing effluent stream from pulp, cellulose pulp or cellulose production is used as the aqueous, alkaline lignin-containing composition.

In a particularly preferred embodiment of the process according to the invention, black liquor from the paper industry, cellulose pulp or cellulose production is used for the production of the aqueous, alkaline lignin-containing composition.

As alkalis or bases for the production of the aqueous, alkaline lignin-containing compositions or for adjusting the pH of the aqueous, alkaline lignin-containing compositions, inorganic bases can in particular be used, e.g. alkali metal hydroxides such as NaOH or KOH, ammonium salts such as ammonium hydroxide and alkali metal carbonates such as sodium carbonate, e.g. In the form of soda. Alkali metal hydroxides, in particular NaOH and KOH, are preferred. The concentration of Inorganic bases in the aqueous, lignin-containing suspension or solution should not exceed 5 mol/L, in particular 4 mol/L, and typically lies in the range from 0.01 to 5 mol/L, in particular in the range from 0.1 to 4 mol/L.

As solid adsorbents, for example basic aluminum oxides, clays, crosslinked organic polymer resins, e.g. crosslinked basic polymer resins or crosslinked cationic polymer resins, active carbon, in particular non-chemically-activated active carbon or chemically pretreated active carbon, e.g. base-impregnated or washed active carbon, are suitable.

The solid adsorbent is preferably selected from crosslinked organic polymer resins, in particular from crosslinked basic organic polymer resins and crosslinked cationic polymer resins, and in particular from active carbon, especially steam-activated active carbon.

The crosslinked basic or cationic organic polymer resins are preferably anion exchangers or anion exchange resins. The preferred anion exchangers or anion exchange resins as a rule have functional groups which are selected from tertiary amino groups, quaternary ammonium groups and quaternary phosphonium groups. Particularly preferably, the anion exchangers used are crosslinked, organic polymer resins which have cationic groups, for example quaternary ammonium groups, quaternary phosphonium groups, imidazolium groups or guanidinium groups, in particular quaternary ammonium groups or imidazolium groups. Suitable anion exchange resins are the anion exchange resins described in WO 2014/006108, to which reference is here made in full.

The base-impregnated active carbon is active carbon which has been pretreated with bases, as defined above. The base-impregnated active carbon is preferably active carbon which has been pretreated with NaOH. For the impregnation, the active carbon is as a rule washed several times with an aqueous solution of the base.

In a particularly preferred embodiment of the process according to the invention, the solid adsorbent is selected from non-chemically activated or base-impregnated or washed active carbons.

The non-chemically activated active carbon is preferably active carbon activated with steam. The steam-activated active carbon is as a rule normal commercial active carbon such as for example CAL® or Aquacarb® 207C from Chemviron Carbon, Norit® ROY 0.8 and Norit® GAC 1240 from Norit or Epibon® A 8×30 or Hydraffin® 30N from Donau Carbon.

The treatment of the lignin-containing suspension or solution with the solid adsorbent, in particular the anion exchange resin or the untreated or the base-impregnated active carbon, is as a rule effected at a temperature in the range from 10 to 100° C., preferably in the range from 10 to 70° C., in particular in the range from 15 to 50° C.

In a preferred embodiment of the process according to the invention, for loading the solid adsorbent, the aqueous, basic lignin-containing composition, which has optionally been treated with alkalis or oxidatively, is passed in a normal manner through an adsorbent system, i.e. through one or more fixed beds of the adsorbent, e.g. through one or more columns which are packed with the adsorbent (e.g. an untreated or base-impregnated active carbon). The passage can be effected both descending and also ascending. The passage is preferably effected at a specific flow rate (specific loading) in the range from 0.2 to 35 bed volumes per hour (BV/h), in particular in the range from 0.5 to 10 BV/h, especially in the range from 1 to 5 BV/h. The passage is preferably effected a linear speed in the range from 0.1 to 50 m/h.

The relative quantity of lignin-containing suspension or solution and solid adsorbent is usually selected such that at least 35% and in particular at least 50% of the 3,3′-dimethoxy-4,4′-dihydroxystilbene (I) contained in the aqueous, alkaline composition is adsorbed by the adsorbent. The quantity of aqueous, alkaline composition is as a rule 1 to 100 times, in particular 2 to 50 times the bed volume. Depending on the degree of adsorption, effluent arriving at the outlet of the adsorbent system, e.g. the column packed with adsorbent, can still contain 3,3′-dimethoxy-4,4′-dihydroxystilbene (I), so that the effluent can optionally also be passed onto a further adsorbent system, e.g. a column packed with adsorbent.

The loading procedure can be followed by a washing step. Usually an aqueous liquid is used for washing the laden adsorbent. An aqueous liquid is understood to be water or a mixture of water with an organic solvent miscible with water, wherein water is the main component of the mixture and in particular 90 vol. % of the mixture. The pH of the aqueous liquid usually lies in the neutral range, i.e. In the range from pH 6 to pH 8. The washing step is as a rule effected at a temperature and at a pressure as defined above for the loading of the adsorbent. If the loading of the adsorbent is effected in an adsorbent system, the aqueous liquid, in particular water, will be passed upwards or downwards through the adsorbent system. The quantity of aqueous liquid, hereinafter also washing water, in this step is usually 1 to 20 times the bed volume, in particular 2 to 10 times the bed volume. The passage of the washing water is as a rule effected at a specific flow rate (specific loading) in the range from 0.5 to 10 BV/h, in particular in the range from 1 to 8 BV/h or a linear speed in the range from 0.1 to 50 m/h. The washing waters thus arising can contain small quantities of 3,3′-dimethoxy-4,4′-dihydroxystilbene (I) and can then be combined with the 3,3′-dimethoxy-4,4′-dihydroxystilbene (I) arising during the loading.

Optionally, following the loading step or in particular following the washing step and before the desorption, the adsorbent can be treated with an aqueous solution of an acid, in particular a mineral acid or an organic sulfonic acid in order to protonate or neutralize the bound anionic 3,3′-dimethoxy-4,4′-dihydroxystilbene. Suitable mineral acids are for example hydrochloric acid, nitric acid, perchloric acid, phosphoric acid or sulfuric acid. Suitable organic sulfonic acids are in particular methanesulfonic acid. A particularly preferred mineral acid is sulfuric acid. Preferably, the aqueous solution of the acid has an acid concentration in the range from 0.01 to 10 mol kg⁻¹, preferably in the range from 0.1 to 5 mol kg⁻¹ in particular 0.1 to 2 mol kg⁻¹.

If the loading of the adsorbent is effected in an adsorbent system, the aqueous dilute acid, optionally after a washing step, will be passed upwards or downwards through the adsorbent system in order in any case to protonate or neutralize bound 3,3′-dimethoxy-4,4′-dihydroxystilbene. The quantity of aqueous dilute acid is usually 0.1 to 10 times the bed volume, in particular 0.5 to 5 times the bed volume. The passage of the aqueous dilute mineral acid is as a rule effected at a specific flow rate (specific loading) in the range from 0.5 to 10 BV/h, in particular in the range from 1 to 8 BV/h.

The treatment with the aqueous dilute aqueous acid can be followed by a further washing step with water. Concerning the quantity of the water and the flow rate, that in the aforesaid washing step applies.

Next, to obtain the 3,3′-dimethoxy-4,4′-dihydroxystilbene (I) the adsorbent is eluted in a manner in itself known. Suitable in particular as eluents are organic solvents, solutions of acids, in particular mineral acids, in organic solvents and solutions of adds, in particular mineral acids, in organic-aqueous solvent mixtures. The nature of the eluent naturally depends on the adsorbent used. In the case of ion exchangers, concerning the elution reference is made to WO 2014/006108. As a rule the procedure followed is that a suitable eluent, for example an organic solvent or a solution of an acid in an organic solvent or a solution of an acid in an organic-aqueous solvent mixture, is passed through the adsorbent system, whereby the bound 3,3′-dimethoxy-4,4′-dihydroxystilbene (I) is desorbed and eluted. If the loading of the adsorbent is effected in an adsorbent system, after the loading and optionally the washing step and/or the treatment with the aqueous acid, an organic solvent or a solution of an acid in an organic solvent or a solution of an acid in an organic-aqueous solvent mixture is passed through the adsorbent system, whereby the bound, optionally neutralized or protonated 3,3′-dimethoxy-4,4′-dihydroxystilbene is desorbed and eluted. The quantity of organic solvent is as a rule 0.1 to 20 times, in particular 0.5 to 15 times, e.g. 1 to 10 times the bed volume. The eluent is as a rule passed at a specific low rate (specific loading) in the range from 0.5 to 20 BV/h, preferably in the range from 0.5 to 10 BV/h, in particular in the range from 1 to 8 BV/h. If an anion exchange resin is used as the solid adsorbent, this must usually be converted into the OH form again, e.g. by treatment with an aqueous solution of an alkali metal hydroxide, e.g. with aqueous NaOH, before the next loading.

The organic solvents used for the elution can be organic solvents miscible with water and organic solvents poorly miscible with water and mixtures thereof.

The organic solvents miscible with water are as a rule organic solvents which are unrestrictedly miscible with water at 22° C. or at least dissolve in water at 22° C. in a quantity of at least 200 g/L. These include in particular dimethyl sulfoxide, acetone, C₁-C₄ alkanols such as methanol, ethanol, isopropanol, n-propanol, 1-butanol, 2-butanol or tert.-butanol, alkane diols such as glycol or 1,4-butanediol, alkyl nitriles such as acetonitrile, but also cyclic ethers such as dioxane, methyltetrahydrofuran or tetrahydrofuran, nitrogen heterocycles such as pyridine or N-methylpyrrolidine and mixtures thereof. Preferably the organic solvents miscible with water are C₁-C₄ alkanols and especially methanol.

The organic solvents poorly miscible with water are as a rule organic solvents which dissolve in water at 22° C. in a quantity of at most 50 g/L. These in particular include aliphatic hydrocarbons such as pentane, hexane, heptane, ligroin, petroleum ether or cyclohexane, halogenated hydrocarbons such as dichloromethane, trichloromethane or tetrachloromethane, aromatic hydrocarbons such as benzene, toluene or xylenes, halogenated aromatic hydrocarbons such as chlorobenzene or dichlorobenzenes, dialkyl ethers such as diethyl ether, methyl tert.-butyl ether or dibutyl ether and mixtures thereof. The organic solvents not miscible with water are preferably aromatic hydrocarbons, in particular toluene or xylenes and mixtures thereof.

Concerning the temperatures and the pressure in the elution, those stated for the loading apply. The elution can be performed both ascending and also descending. The elution can be performed in the same direction as the loading or oppositely thereto.

Optionally, before the elution step, the water present in the pores and between the adsorbent particles or, if a water-insoluble organic solvent was used for the elution, the water-insoluble organic solvent remaining in the pores and between the adsorbent particles is removed with a water-miscible organic solvent such as methanol or ethanol. For this, the water-miscible organic solvent is passed upwards through the adsorbent system. The quantity of water-miscible organic solvent is usually 0.5 to 10 times, in particular 1 to 5 times the bed volume. The water-miscible solvent is preferably passed at a specific flow rate (specific loading) in the range from 0.5 to 10, in particular 1 to 8 bed volumes per hour.

The elution can be followed by a further washing step in order to remove impurities that are possibly present.

The adsorbent system can be operated batchwise and then has one or more stationary fixed beds packed with adsorbent, e.g. 2, 3 or 4 connected in series. It can also be operated continuously and then as a rule has 5 to 50 and in particular 15 to 40 adsorbent beds which can for example be a component of a “True Moving Bed” system (see K. Takeuchi J. Chem. Eng. Jpn., 1978, 11 pp. 216-220), a “Continuous Circulating Annular” system (see J. P. Martin, Discuss. Faraday Soc. 1949, p. 7) or a “Simulated Moving Bed” system, as for example described in U.S. Pat. No. 2,985,589 and WO 01/72689 and by G. J. Rossiter et al. Proceedings of AlChE Conference, Los Angeles, Calif., November 1991 or H. J. Van Walsem et al., J. Biochtechnol. 1997, 59, p. 127.

The eluent arising in the elution contains 3,3′-dimethoxy-4,4′-dihydroxystilbene (I), which can in this way be obtained in concentrated form.

The eluate obtained in the elution is worked up in the usual manner to obtain the 3,3′-dimethoxy-4,4′-dihydroxystilbenes. If the eluate contains acid, this will as a rule be removed first, for example by an aqueous extractive workup, or neutralized by addition of base and the salts thereby formed removed. Optionally, the eluate can be concentrated beforehand, e.g. by removal of the solvent in a normal evaporator system. The condensate thus arising can be reused, for example in a subsequent elution.

In this manner, a 3,3′-dimethoxy-4,4′-dihydroxystilbene-containing crude product is obtained, which possibly contains other low molecular weight components and possibly other components of the aqueous composition used, for example guaiacol, vanillin, acetovanillone or lignin.

In a preferred embodiment of the process according to the invention, the 3,3′-dimethoxy-4,4′-dihydroxystilbene-containing eluate or crude product obtained after the desorption or elution is subjected to a further purification step. The purification step can for example comprise a rectification, crystallization or a liquid chromatographic separation. The 3,3′-dimethoxy-4,4′-dihydroxystilbene-containing eluate or crude product is preferably purified by distillation or crystallized.

The invention is illustrated in more detail on the basis of the examples described below. However, the examples should not be understood as limiting the invention.

In the following examples the following abbreviations are used:

BV stands for bed volume;

DI water stands for deionized (demineralized) water.

### EXAMPLES

### I) Analysis

The content of 3,3′-dimethoxy-4,4′-dihydroxystilbene (I) and other organic components of the aqueous lignin-containing compositions used was determined by high performance liquid chromatography (HPLC). As the stationary phase, the column Chromolith® High Resolution RP18e from Merck (length: 100 mm, diameter 4.6 mm) was used. The analysis temperature was 25° C. In this, two mobile phases were used: HPLC water with 0.1 wt. % perchloric acid as mobile phase A; acetonitrile as mobile phase B.

II) Adsorption and Desorption of 3,3′-Dimethoxy-4,4′-dihydroxystilbene (i) Using Active Carbon and Ion Exchange Resins

### Example II.1: Adsorption and Desorption of 3,3′-dimethoxy-4,4′-dihydroxystilbene (I) Using the Active Carbon Norit® ROY 0.8 from Norit

### Active Carbon Used:

In the experiment, the active carbon Norit® ROY 0.8 from Norit was used. This active carbon is a coal-based extrudate and after steam activation is washed several times with sodium hydroxide solution (aqueous NaOH). The bulk density of the active carbon is 400 g/L. The active carbon has a moisture content of max. 5%.

### Lignin-Containing Composition Used:

As the lignin-containing composition, black liquor (thin liquor) from cellulose pulp production was used. For the experiment, the black liquor was filtered with a metal filter (filter pore size=90 micrometers). The HPLC analysis of the filtered black liquor gave a 3,3′-dimethoxy-4,4′-dihydroxystilbene (I) content of 490 mg/kg.

### Experimental Method:

A glass column with an internal diameter of 15 mm and a height of 255 mm was set up and filled with the active carbon Norit® ROY 0.8 to ca. 95% fill level. The bed volume (BV) was ca. 43 mL. The active carbon was washed with ca. 10 BV of DI water at a speed of ca. 5 BV/h downwards.

For the absorption of the organic components, ca. 12 BV of filtered black liquor was passed through the column at a speed of ca. 2 BV/h downwards. The column outflow was collected in fractions. The fractions were analyzed for organic components. The loading of 3,3′-dimethoxy-4,4′-dihydroxystilbene (I) achieved was about 0.04 mol/L. After this, the active carbon was washed with 5 BV of DI water at a speed of ca. 2 BV/h downwards.

After the washing step, an acid washing was performed in order to protonate the absorbed 3,3′-dimethoxy-4,4′-dihydroxystilbene (I). For this, ca. 1 BV of 5 percent sulfuric acid was passed through the column at a speed of ca. 2 BV/h downwards. After this, the active carbon was washed with ca. 5 BV of DI water at a speed of ca. 2 BV/h downwards.

In order to eliminate the water between the active carbon particles and in the pores present therein, after the washing step, ca. 2 BV of pure methanol was passed through the column at a speed of ca. 2 BV/h upwards.

For desorption of the adsorbed 3,3′-dimethoxy-4,4′-dihydroxystilbenes, firstly ca. 2 BV of a mixture of methanol and toluene in a mass ratio of 9:1 was passed through the column at a speed of 2 BV/h upwards. Next, for further desorption, ca. 3 BV of pure toluene was passed through the column at a speed of 2 BV/h upwards. In the desorption step, the column outflow was collected in one fraction. This fraction was analyzed for its content of 3,3′-dimethoxy-4,4′-dihydroxystilbene. The desorption level of 3,3′-dimethoxy-4,4′-dihydroxystilbene was about 3 to 4%.

After the desorption step, ca. 1 BV of pure methanol were passed through the column at a speed of ca. 2 BV/h upwards.

After the methanol washing, the active carbon was washed with ca. 10 BV of DI water at a speed of ca. 5 BV/h upwards.

AU steps of the experiment were performed at room temperature.

### Example II.2: Adsorption and Desorption of 3,3′-dimethoxy-4,4′-dihydroxystilbene (I) Using the Active Carbon Aquacarb™ 207C from Chemviron Carbon

### Active Carbon Used:

In the experiment, the active carbon Aquacarb™ 207C from Chemviron Carbon was used. This active carbon is a coconut-based granulated active carbon activated with steam. The bulk density of the active carbon is 450 g/L. The active carbon has a moisture content of max. 10% auf.

### Lignin-Containing Composition Used:

As the lignin-containing composition, black liquor (thin liquor) from cellulose pulp product was used. For the experiment the black liquor was filtered with a metal filter (filter pore size=90 micrometers). The HPLC analysis of the filtered black liquor gave the following concentrations of the organic components: 457 mg/kg vanillin, 349 mg/kg acetovanillone, 506 mg/kg guaiacol and 308 mg/kg 3,3′-dimethoxy-4,4′-dihydroxy-stilbene.

### Experimental Method:

A glass column with an Internal diameter of 15 mm and a height of 255 mm was set up and filled with the active carbon Aquacarb™ 207C to ca. 95% fill level. The bed volume (BV) was ca. 43 mL. The active carbon was firstly washed with ca. 10 BV of DI water at a speed of ca. 5 BV/h downwards.

For the adsorption of the organic components, ca. 12 BV of filtered black liquor were passed through the column at a speed of ca. 2 BV/h downwards. The column outflow was collected in fractions. The fractions were analyzed for organic components. The achieved loading of the Individual organic components on the active carbon was: 0.02 mol/L vanillin, 0.01 mol/L acetovanillone, 0.03 mol/L guaiacol and 0.01 mol/L 3,3′-dimethoxy-4,4′-dihydroxystilbene. After this, the active carbon was washed with ca. BV of DI water at a speed of ca. 2 BV/h downwards.

The washing step was followed by an acid washing in order to protonate the adsorbed organic anions. For this, ca. 1 BV of 5 percent sulfuric acid was passed through the column at a speed of ca. 2 BV/h downwards. After this, the active carbon was washed with ca. 5 BV of DI water at a speed of ca. 2 BV/h downwards.

In order to eliminate the water between the active carbon particles and in the pores present therein, after the washing step ca. 2 BV of pure methanol was passed through the column a speed of ca. 2 BV/h upwards.

For the desorption of the adsorbed organic components, firstly ca. 2 BV of a mixture of methanol and toluene in the mass ratio of 1:1 was passed through the column at a speed of 2 BV/h upwards. Next, for further desorption ca. 3 BV of pure toluene was passed through the column at a speed of 2 BV/h upwards. In the desorption step, the column outflow was collected in one fraction. This fraction was analyzed for the content of the organic components. The achieved desorption level of the individual organic components was: 89% for vanillin, 95% for acetovanillone, 89% for guaiacol and 8% for 3,3′-dimethoxy-4,4′-dihydroxystilbene.

After the desorption step, ca. 1 BV of pure methanol was passed through the column at a speed of ca. 2 BV/h upwards.

After the methanol washing, the active carbon was washed with ca. 10 BV of DI water at a speed of ca. 5 BV/h upwards.

All steps of the experiment were performed at room temperature.

### Example II.3 to II.6: Adsorption and Desorption of 3,3′-dimethoxy-4,4′-dihydroxystilbene (I) Using Various Anion Exchange Resins

### Anion Exchange Resins Used:

In the experiment the following anion exchange resins were used:

| | | Ion exchanger | | Ion exchange |
|---|---|---|---|---|
| Example | Producer | brand name | Functional group | capacity |
| II.3 | Dow | Dowex | Trimethylammonium | 1.10 mol/Lᵣₑₛᵢₙ |
| | | Monosphere | groups | |
| | | 550A OH | | |
| II.4 | Dow | Amberlite | Dimethylethanol- | 1.25 mol/Lᵣₑₛᵢₙ |
| | | IRA 410 CI | ammonium groups | |
| II.5 | Lanxess | Lewatit | Tripropylammonium | 0.60 mol/Lᵣₑₛᵢₙ |
| | | Ionac SR7 | groups | |
| II.6 | Lanxess | Lewatit | Tributylammonium | 0.55 mol/Lᵣₑₛᵢₙ |
| | | Ionac SR6 | groups | |

### Lignin-Containing Composition Used:

As the lignin-containing composition, black liquor (thin liquor) from cellulose pulp production was used. For the experiment, the black liquor was filtered with a metal filter (filter pore size=90 micrometers). The HPLC analysis of the filtered black liquor gave the following concentrations of the organic component: 447 mg/kg vanillin, 268 mg/kg acetovanillone, 460 mg/kg guaiacol and 490 mg/kg 3,3′-dimethoxy-4,4′-dihydroxy-stilbene (I).

### Experimental Method:

A glass column with an internal diameter of 15 mm and a height of 255 mm was set up and filled with the respective anion exchange resins to ca. 90% fill level. The bed volume (BV) was ca. 40 mL. To convert the anion exchange resins into the OH form, these, with the exception of the anion exchange resin Dowex Monosphere 550A OH, were washed firstly with a 4 percent NaOH solution and then with ca. 10 BV of DI water at a speed of ca. 5 BV/h downwards.

For the adsorption of the organic components, ca. 6 BV of filtered black liquor were passed through the column at a speed of 2 to 4 BV/h downwards. The column outflow was collected in fractions. The fractions were analyzed for organic components. The achieved loading of organic components on the respective anion exchange resins were for:

Dowex Monosphere 550A OH: 0.07 mol/Lᵣₑₛᵢₙ,
Amberlite IRA 410 Cl: 0.07 mol/Lᵣₑₛᵢₙ,
Lewatit Ionac SR7: 0.05 mol/Lᵣₑₛᵢₙ,
Lewatit Ionac SR6: 0.05 mol/Lᵣₑₛᵢₙ.

After this, the active carbon was washed with ca. 5 BV of DI water at a speed of ca. 2 BV/h downwards.

For the desorption of the adsorbed organic components, ca. 5 BV of a mixture of 5% sulfuric acid, 45% methanol and 50% DI water were passed through the column at a speed of 2 to 4 BV/h upwards. In the desorption step, the column outflow was collected in one fraction. This fraction was analyzed for the content of the organic components. The desorption level of the individual organic components achieved is listed below:

| | | Desorption level | | | |
|---|---|---|---|---|---|
| | Ion | | Aceto- | | Stilbene |
| Example | exchanger | Vanillin: | vanillone: | Guaiacol: | derivative (I): |
| II.3 | Dowex | ca. 54% | ca. 50% | ca. 60% | ca. 22% |
| | Monosphere | | | | |
| | 550A OH | | | | |
| II.4 | Amberlite | ca. 54% | ca. 52% | ca. 62% | ca. 28% |
| | IRA 410 Cl | | | | |
| II.5 | Lewatit | ca. 61% | ca. 53% | ca. 27% | ca. 13% |
| | Ionac SR7 | | | | |
| II.6 | Lewatit | ca. 48% | ca. 38% | ca. 23% | ca. 7% |
| | Ionac SR6 | | | | |

After the desorption step, the active carbon was washed with ca. 10 BV of DI water at a speed of ca. 2 BV/h downwards.

For regeneration of the anion exchange resin, ca. 5 BV of a 4 to 10 percent NaOH solution was passed through the column at a speed of ca. 2 to 4 BV/h upwards and it was then washed with ca. 10 BV of DI water at a speed of ca. 4 BV/h downwards.

All steps of the experiment were performed at room temperature.

### III) Smelling Strip Test:

In order to test the quality and intensity of the odor of 3,3′-dimethoxy-4,4′-dihydroxy-stilbene or (E)-3,3′-dimethoxy-4,4′-dihydroxystilbene, smelling strip tests were performed.

These show that the compound (E)-3,3′-dimethoxy-4,4′-dihydroxystilbene both after storage in the dark and also after exposure to light evokes a sweet (aromatized) odor impression, in particular a typical vanilla-like odor impression which is reminiscent of sweetened milk.

This typical vanilla-like odor impression intensifies significantly when (E)-3,3′-dimethoxy-4,4′-dihydroxystilbene is exposed to sunlight. The intensification of the vanilla-like odor impression cannot be quantified in the odor laboratory, but is perceptible to anyone.

1.-15. (canceled)

16. Use of the compound 3,3′-dimethoxy-4,4′-dihydroxystilbene (I) 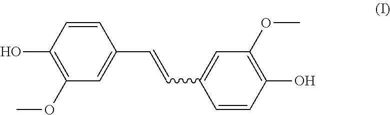 [SMILES: COc1cc(C=Cc2ccc(O)c(OC)c2)ccc1O]
as an odorous substance

17. Use as claimed in claim 16, wherein the compound (I) is present as the E isomer (I-E) 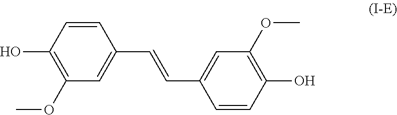 [SMILES: COc1cc(/C=C/c2ccc(O)c(OC)c2)ccc1O]
or as an E/Z isomer mixture which predominantly contains the E isomer (I-E).

18. Use of the compound (I), as defined in claim 16 for developing a vanilla odor under the action of light.

19. Use as claimed in claim 16, wherein the compound (I) is a component of a composition which additionally contains a carrier.

20. Use as claimed in claim 19, wherein the composition is selected from washing powders, laundry conditioners, cleansing agents, fragrance-containing hygiene products, fragrance dispensers and perfumes.

21. Use as claimed in claim 19, wherein the compound (I) is a component of a washing powder and/or a laundry conditioner.

22. A fragrance composition and/or a odorant material comprising 3,3′-dimethoxy-4,4′-dihydroxystilbene (I) 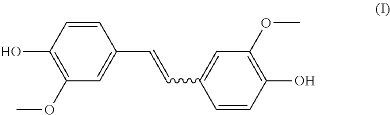 [SMILES: COc1cc(C=Cc2ccc(O)c(OC)c2)ccc1O]
and a carrier, wherein the fragrance composition and/or the odorant material contains the compound (I) in a quantity which imparts an odor to the fragrance composition and/or the odorant material or modifies the odor of the fragrance composition and/or the odorant material.

23. A method for imparting or modifying an odor of a composition, comprising adding 3,3′-dimethoxy-4,4′-dihydroxystilbene (I) 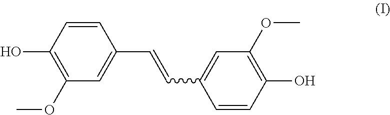 [SMILES: COc1cc(C=Cc2ccc(O)c(OC)c2)ccc1O]
to the composition in a quantity which imparts an odor to the composition or modifies the odor of the composition.

24. A method for obtaining 3,3′-dimethoxy-4,4′-dihydroxystilbene (I) from an aqueous, alkaline lignin-containing composition, characterized in that the aqueous, basic lignin-containing composition, which has optionally been treated with alkalis or oxidatively, is treated with a solid adsorbent, the adsorbent is separated from the aqueous, alkaline lignin-containing composition and then for the obtention of the 3,3′-dimethoxy-4,4′-dihydroxystilbene (I) the adsorbent is treated with an eluent, whereby a 3,3′-dimethoxy-4,4′-dihydroxystilbene-containing eluate is obtained.

25. The method as claimed in claim 24, wherein the eluent is selected from organic solvents.

26. The method as claimed in claim 24, wherein the eluent is selected from C₁-C₄ alkanols, aromatic hydrocarbons, and mixtures thereof.

27. The method as claimed in claim 24, wherein the 3,3′-dimethoxy-4,4′-dihydroxystilbene-containing eluate is subjected to a further purification step.

28. The method as claimed in claim 24, wherein the aqueous, alkaline lignin-containing composition is black liquor from the paper industry, cellulose pulp or cellulose production.

29. The method as claimed in claim 24, wherein the solid adsorbent is selected from crosslinked basic or cationic, organic polymer resins and active carbon.

30. The method as claimed in claim 24, wherein the solid adsorbent is selected from active carbon.
